(19) United States
(12) Patent Application Publication
(10) Pub. No.: US 2026/0060563 A1
(43) Pub. Date: Mar. 5, 2026

(54) ADAPTIVE PHOTOELECTRIC HEART RATE DETECTION METHOD, WEARABLE ELECTRONIC DEVICE AND STORAGE MEDIUM

(71) Applicant: PETAVERSE PTE. LTD., Singapore (SG)

(72) Inventor: Bei HONG, Shanghai (CN)

(73) Assignee: PETAVERSE PTE. LTD., Singapore (SG)

(21) Appl. No.: 19/382,799

(22) Filed: Nov. 7, 2025

(51) Int. Cl. A61B 5/024; A61B 5/00

(52) U.S. Cl. CPC A61B 5/02427; A61B 5/725; A61B 5/7264; A61B 5/7271; A61B 2560/0462

(57) ABSTRACT

Disclosed are an adaptive photoelectric heart rate detection method based on hair characteristics, a wearable electronic device and a storage medium, including: collecting the signal attenuation rate of an object's hair wearing a photoelectric sensor; selecting a target wave band of the light source of the photoelectric sensor based on the signal attenuation rate, and selecting a target drive current value of the photoelectric sensor based on the signal attenuation rate; driving the light source to emit light waves according to the target drive current value and the target wave band; collecting the optical signal received by the detector of the photoelectric sensor, and detecting the heart rate value of the object based on the frequency value of the optical signal. This method can adjust the light source intensity to adapt to dense hair conditions, thereby eliminating hair artifact interference and improving the accuracy and universality of pet health monitoring.

### TECHNICAL FIELD

This application relates to the field of photoelectric sensing technology, in particular to an adaptive photoelectric heart rate detection method based on hair characteristics, a wearable electronic device and a storage medium.

### BACKGROUND

Existing photoplethysmography heart rate sensors are primarily used for human health monitoring, with most structures based on single-wave band light sources and fixed-intensity photoelectric detection. However, in pet scenarios, due to animals generally having dense and variably colored hair, the following issues often arise:
1. Light blockage attenuation: hair obstructs the light path, causing a significant decrease in the optical signal effectively transmitted to the skin surface;
2. Scattering noise: the random arrangement and movement of hair result in incident light reflection and scattering, increasing noise;
3. Baseline drift and low detection rate: traditional devices perform well under human bare-skin conditions, but under pet hair conditions, both heart rate detection rate and accuracy significantly decline.

### SUMMARY

Based on this, it is necessary to address the above technical problems by providing an adaptive photoelectric heart rate detection method based on hair characteristics, a wearable electronic device, and a storage medium, which can automatically adjust the light source intensity of the photoelectric sensor to adapt to dense hair conditions, thereby eliminating hair artifact interference and improving the accuracy and universality of pet health monitoring.

This application provides an adaptive photoelectric heart rate detection method based on hair characteristics, including the following steps of: collecting a signal attenuation rate of the hair of an object wearing a photoelectric sensor; selecting a target wave band of a light source of the photoelectric sensor based on the signal attenuation rate, and selecting a target drive current value of the photoelectric sensor based on the signal attenuation rate; driving the light source of the photoelectric sensor to emit light waves according to the target drive current value and the target wave band; and collecting optical signals received by a detector of the photoelectric sensor, and detecting a heart rate value of the object based on a frequency value of the optical signals.

Preferably, the step of collecting a signal attenuation rate of the hair of an object wearing a photoelectric sensor includes: providing the photoelectric sensor with a certain light wavelength and a drive current, and collecting a signal intensity received by the photoelectric sensor; obtaining a standard reflected signal intensity; and determining the signal attenuation rate based on the collected signal intensity and the standard reflected signal intensity.

Preferably, the signal attenuation rate is calculated by providing the photoelectric sensor with a green light wave band and a certain drive current; the step of selecting a target wave band of a light source of the photoelectric sensor based on the signal attenuation rate includes: when the signal attenuation rate is less than a first set value, selecting the target wave band of the light source of the photoelectric sensor as a wave band combining green light with an infrared wavelength, prioritizing green light; when the signal attenuation rate is greater than or equal to the first set value and less than a second set value, selecting the target wave band of the light source of the photoelectric sensor as a wave band combining infrared with a green light wavelength, prioritizing infrared; when the signal attenuation rate is greater than or equal to the second set value and less than a third set value, selecting the target wave band of the light source of the photoelectric sensor as a wave band combining infrared with a red light wavelength, prioritizing infrared; and when the signal attenuation rate is greater than or equal to the third set value, selecting the target wave band of the light source of the photoelectric sensor as a wave band combining infrared with a red light wavelength, prioritizing infrared, wherein when the signal attenuation rate is greater than or equal to the third set value, the optical signals of the photoelectric sensor require multiple repeated samplings; and wherein the first set value is greater than 0 and less than the second set value, the second set value is less than the third set value, and the third set value is less than 1.

Preferably, there are multiple photoelectric sensors, and the step of selecting a target drive current value of the photoelectric sensor based on the signal attenuation rate includes: obtaining a lower limit value and an upper limit value of the signal intensity of the photoelectric sensor; obtaining a starting drive current value and a step size value of the drive current of the photoelectric sensor; providing the drive current to each photoelectric sensor based on the starting drive current value, and collecting the signal intensity of each photoelectric sensor, wherein if the signal intensity of any photoelectric sensor is not greater than the lower limit value, the drive current of each photoelectric sensor is gradually increased according to the step size value until the signal intensity of at least one photoelectric sensor exceeds the lower limit value, and a drive current value at this time is recorded as OptCrMin; continuing to gradually increase the drive current of each photoelectric sensor according to the step size value until the signal intensity of all photoelectric sensors is greater than the lower limit value and less than the upper limit value, and recording the drive current value at this time as OptCrMax; and selecting the target drive current value based on the OptCrMin and the OptCrMax.

Preferably, there are two photoelectric sensors, and the step of selecting the target drive current value based on the OptCrMin and the OptCrMax includes: selecting a mean value of the OptCrMin and the OptCrMax as the target drive current value.

Preferably, the step of detecting a heart rate value of the object based on a frequency value of the optical signals includes: performing band-pass filtering on the optical signals to obtain filtered signals; selecting K frequency point signals with a highest amplitude value from the filtered signal; performing frequency multiplication interference removal processing on the K frequency point signals to obtain M frequency point signals; screening out effective frequency points from the M frequency point signals; and converting frequency values of the screened effective frequency points into the heart rate value of the object; where K and M are both positive integers.

Preferably, the step of screening out effective frequency points from the M frequency point signals includes: identifying whether there is an interval with consecutive frequency values among the M frequency point signals; if one or more intervals with consecutive frequency values exist, selecting multiple frequency points in the interval with a highest frequency value as multiple effective frequency points; the step of converting frequency values of the screened effective frequency points into the heart rate value of the object includes: performing weighted average processing on the frequency values of multiple effective frequency points to obtain an average frequency; and converting the average frequency into the heart rate value of the object.

Preferably, the adaptive photoelectric heart rate detection method further includes:
if no intervals with consecutive frequency values exist, treating all M frequency point signals as the effective frequency points;
performing weighted average processing on the frequency values of the M frequency point signals to obtain the average frequency; and
converting the average frequency into the heart rate value of the object.

This application further provides an adaptive photoelectric heart rate detection device based on hair characteristics, including: a collection module for collecting a signal attenuation rate of the hair of an object wearing a photoelectric sensor; a parameter selection module for selecting a target wave band of a light source of the photoelectric sensor based on the signal attenuation rate and determining a target drive current value of the photoelectric sensor; a drive module for driving the light source of the photoelectric sensor to emit light waves based on the target drive current value and the target wave band; a detection module for collecting optical signals received by a detector of the photoelectric sensor and detecting a heart rate value of the object based on a frequency value of the optical signals.

This application further provides a wearable electronic device, wherein the wearable electronic device is equipped with a reflective photoelectric sensor including a microprocessor and a memory, as well as a computer program stored in the memory and executable on a processor, wherein the processor, when executing the computer program, implements the steps of any of the above methods.

This application further provides a storage medium, having a computer program stored thereon, wherein the computer program, when executed by a processor, implements the steps of any of the above methods.

In the above adaptive photoelectric heart rate detection method based on hair characteristics, the wearable electronic device, and the storage medium, the method includes: collecting the signal attenuation rate of the hair of an object wearing a photoelectric sensor; selecting the target wave band of the light source of the photoelectric sensor based on the signal attenuation rate, and selecting the target drive current value of the photoelectric sensor based on the signal attenuation rate; controlling the light source of the photoelectric sensor to emit light waves according to the target wave band; driving the light source of the photoelectric sensor to emit light waves based on the target drive current value and the target wave band; collecting the optical signals received by the detector of the photoelectric sensor, and detecting the heart rate value of the object based on the frequency value of the optical signals. Therefore, since different objects (such as pets) have varying hair density characteristics, resulting in different signal attenuation rates, this application dynamically selects the wave band and drive current value of the photoelectric sensor's light source based on the signal attenuation rate of the hair of the specific object wearing the photoelectric sensor, thereby driving the light source to emit light waves. This enables automatic adjustment of the light source intensity of the photoelectric sensor to adapt to dense hair conditions, and subsequently detects the pet's heart rate value based on the frequency value of the optical signals received by the photoelectric sensor, effectively eliminating hair artifact interference and improving the accuracy and universality of pet health monitoring.

### BRIEF DESCRIPTION OF DRAWINGS

FIG. 1 is a schematic flowchart of an adaptive photoelectric heart rate detection method based on hair characteristics in an embodiment;

FIG. 2 is a schematic diagram of the data calculation process of an adaptive photoelectric heart rate detection method based on hair characteristics in an embodiment;

FIG. 3 is a structural schematic diagram of an adaptive photoelectric heart rate detection device based on hair characteristics in an embodiment.

### DESCRIPTION OF EMBODIMENTS

To make the objectives, technical solutions, and advantages of this application clearer, the invention is further described in detail below with reference to the accompanying drawings and embodiments. It should be understood that the specific embodiments described herein are only intended to explain this application and not to limit it.

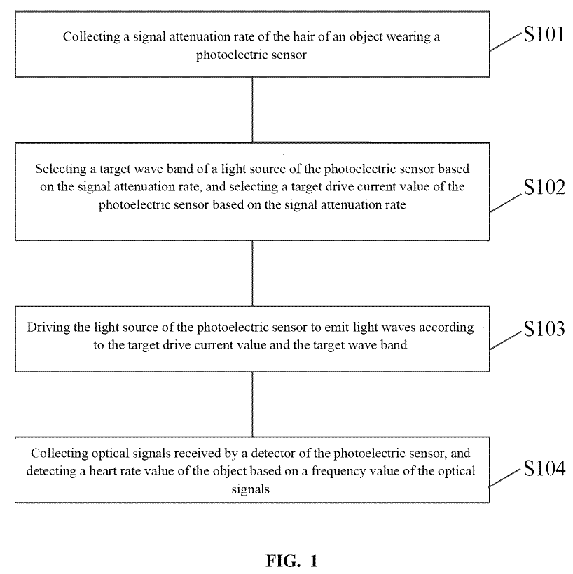

This application provides an adaptive photoelectric heart rate detection method based on hair characteristics. As shown in FIG. 1, in one embodiment, the adaptive photoelectric heart rate detection method based on hair characteristics includes the following steps:

S101: the signal attenuation rate of the hair of an object wearing a photoelectric sensor is collected.

In this embodiment, the object wearing the photoelectric sensor may be a pet, such as a cat, dog, or rabbit. The signal attenuation rates of different pets' fur colors and hair lengths vary under light of different wavelengths.

In one example, the above process of collecting the signal attenuation rate of the hair of an object wearing a photoelectric sensor includes: providing the photoelectric sensor with a specific light wavelength and a drive current, and collecting the signal intensity received by the photoelectric sensor; obtaining the standard reflected signal intensity; and determining the signal attenuation rate based on the collected signal intensity and the standard reflected signal intensity.

In this example, a feature library of attenuation rates for fur color and hair length under different light wavelengths is established. Specifically, since animal fur and hair exhibit different absorptivity, transmittance, and reflectivity for light of varying wave bands, the signal attenuation rate can be calculated by comparing the actually received signal intensity with the standard reflected signal intensity under a given light wavelength and a drive current. This allows obtaining numerical values of signal attenuation rates for multiple wavelengths.

Specifically, the signal attenuation rate is calculated using the following formula:

$${DR}_{wave Length} = 1 - {Sig}_{wave Length} / StdRflCrv ( wave Length , Current ) ;$$

Here, DR_{waveLength} represents the signal attenuation rate, Sig_{waveLength} represents the collected signal intensity, and StdRflCrv(waveLength, Current) represents the standard reflected signal intensity.

The specific process for obtaining the standard reflected signal intensity StdRflCrv(waveLength, Current) is as follows:

First, the device and optical path parameters are calibrated. The optical path calibration design involves using high-reflectivity materials, such as mirrors, surfaces coated with reflective paint, or smooth surfaces covered with flat aluminum foil. Here, the mirror is placed approximately 2 mm-4 mm away from the LED and photodiode (simulating the optical path distance between the LED and sensor to the animal's skin and tissue during actual device use, accounting for the device housing and compressed hair).

For LED devices emitting multiple wave bands, such as green, red, and infrared light, the drive current is gradually increased starting from the minimum drive current. The minimum drive current is typically 1 mA-2 mA.

The correspondence between the drive current and the received signals for the photodiode under different light wavelengths are measured, and the saturation drive current is determined. At this point, the photodiode's received signal reaches its maximum value, and further increasing the LED's drive current will no longer increase the photodiode's signal value.

A mapping relationship is established for {wavelength, drive current, received signal value}. Thus, for each wavelength, a reference signal intensity function can be defined over the interval [minimum drive current, saturation drive current], denoted as: StdRflCrv(waveLength, Current).

S102, the target wave band of the light source for the photoelectric sensor is selected based on the signal attenuation rate, and the target drive current value for the photoelectric sensor is determined according to the signal attenuation rate.

In this embodiment, the target wave band of the light source for the photoelectric sensor is selected based on the collected signal attenuation rate. The target wave band can be a combination of different sampling wave bands. Additionally, determined by the characteristics of the photodiode components, signals that are too small are more susceptible to background noise interference, while signals that are too large may trigger signal saturation in the photoelectric sensor, leading to data distortion. Therefore, by obtaining the actual signal attenuation rate, the optimal drive current value for the photodiode of the photoelectric sensor can be inversely deduced. After adaptive adjustment, the received signal intensity on the photodiode can be maintained within the optimal range, achieving both a high signal-to-noise ratio and avoiding data saturation distortion.

In one example, the signal attenuation rate is calculated by providing the photoelectric sensor with the green light wave band and a certain drive current. The selection of the target wave band for the light source of the photoelectric sensor based on the signal attenuation rate includes: when the signal attenuation rate is less than the first set value, the target wave band for the light source of the photoelectric sensor is selected as green light combined with infrared wavelength, prioritizing green light; when the signal attenuation rate is greater than or equal to the first set value but less than the second set value, the target wave band for the light source of the photoelectric sensor is selected as infrared combined with green light wavelength, prioritizing infrared; when the signal attenuation rate is greater than or equal to the second set value but less than the third set value, the target wave band is selected as infrared combined with red light wavelength, prioritizing infrared; when the signal attenuation rate is greater than or equal to the third set value, the target wave band is selected as infrared combined with red light wavelength, prioritizing infrared, and the optical signal of the photoelectric sensor requires multiple repeated samplings. The first set value is greater than 0 and less than the second set value, the second set value is less than the third set value, and the third set value is less than 1.

In this example, the first set value could be 40%, the second set value could be 60%, and the third set value could be 80%. Of course, the first, second, and third set values can be adjusted according to actual needs and are not limited to these ratios of 40%, 60%, and 80%.

As described above, a characteristic library of hair thickness (5 mm-50 mm) and different hair colors (light, brown, dark brown, black) for the attenuation rates of different light wavelengths was established through practical experiments. It was found in experiments that since the signal attenuation rate of green light is generally higher than that of red and infrared light, observing the attenuation rate DRgreen of green light can optimize the combination strategy of different wavelength light sources during actual sampling:
(1) DRgreen<40%, with low absorptivity, typically indicating light-colored short hair, sampling can be performed using green light combined with infrared wavelength, prioritizing green light;
(2) 40%≤DRgreen<60%, medium absorptivity, typically light-colored long fur or medium-colored medium-length fur, sampling can be performed using infrared combined with green light wavelength, with infrared prioritized;
(3) 60%≤DRgreen<80%, high absorptivity, typically medium-colored long fur or dark-colored short fur, sampling can be performed using infrared combined with red light wavelength, with infrared prioritized;
(4) DRgreen≥80%, extremely high absorptivity, typically dark-colored medium-length fur, sampling can be performed using infrared combined with red light wavelength, with infrared prioritized for repeated sampling.

In one example, the number of photoelectric sensors is multiple. The process of selecting the target drive current value for the photoelectric sensor based on the signal attenuation rate includes: obtaining the lower limit value and upper limit value of the signal intensity of the photoelectric sensor; acquiring the starting drive current value and step size value of the drive current for the photoelectric sensor; providing drive current to each photoelectric sensor based on the starting drive current value and collecting the signal intensity of each photoelectric sensor. If the signal intensity of any photoelectric sensor does not exceed the lower limit value, the drive current for each photoelectric sensor is gradually increased according to the step size value until at least one photoelectric sensor's signal intensity exceeds the lower limit value, at which point the drive current value is recorded as OptCrMin. The drive current for each photoelectric sensor continues to be increased stepwise according to the step size value until the signal intensity of all photoelectric sensors exceeds the lower limit value and remains below the upper limit value, at which point the drive current value is recorded as OptCrMax. The target drive current value is then selected based on OptCrMin and OptCrMax.

Preferably, the number of photoelectric sensors is two, and selecting the target drive current value based on OptCrMin and OptCrMax includes: selecting the average of OptCrMin and OptCrMax as the target drive current value.

Specifically, through multiple experiments, the lower limit of the optimal signal intensity range for the photoelectric sensor, OptSigThMin, is 40-60% of the sensor's saturation value, while the upper limit, OptSigThMax, is 60˜80% of the saturation value.

Among these, the method for adaptively adjusting the drive current is as follows:

Step 1: each time, two photoelectric sensors symmetrically aligned with the LED light bead are activated as the central optical paths to receive signals.

Step 2: the minimum drive current value of the device is recorded as CrMin_{waveLength}, the starting drive current value for adaptive adjustment is CrStart_{waveLength}, and the adaptive adjustment step size value is CrStep_{waveLength}. Then:

$${CrStart}_{waveLength} = {CrMin}_{waveLength} / {DR}_{waveLength} ;$$

$${CrStep}_{waveLength} = {CrMin}_{waveLength} / {DR}_{waveLength} / 3.$$

Step 3: by using CrStep_{waveLength} as the step size value, the drive current is gradually increased until the signal intensity of one of the two photoelectric sensors falls within the [OptSigThMin, OptSigThMax] interval, and the drive current at this point is recorded as OptCrMin.

Step 4: by continually using CrStep_{waveLength} as the step size value, the drive current is gradually increased until the signal intensities of both photoelectric sensors exceed OptSigThMax, and the drive current at this point is recorded as OptCrMax. If both signal intensities do not exceed OptSigThMax when the drive current reaches the maximum allowable value for the LED device, then the maximum allowable drive current of the device is taken as OptCrMax.

Step 5: The drive current during formal sampling is:

$$OptCrSmpl = ( OptCrMin + OptCrMax ) / 2 ;$$

That is, the target drive current value is OptCrSmpl.

Additionally, simultaneously collecting data from both photodiodes can also detect the wearing status of the device. When the signals from the two paths differ significantly, the user is reminded to press the photoelectric sensor firmly during wear.

By combining wavelength-specific sampling strategies with adaptive drive current adjustment, the LED's on-off timing and current are optimized, ensuring sampling quality while minimizing energy consumption.

S103: the light source of the photoelectric sensor to emit light waves according to the target drive current value and the target wave band.

Specifically, the sensor type of the photoelectric sensor is a reflective photoelectric sensor. The light source adopts a multi-wave band combination. The multi-wave bands include green light, red light, and infrared wave bands. The detector of the photoelectric sensor uses 2-6 photodiodes, arranged as follows: 2 bilateral symmetric arrangements, and 4-6 surrounding arrangements, with a light shield added to the surrounding structure to group and partition the photodiodes for signal mutual correction. The wearing method of the photoelectric sensor: a compression-type wearing method is adopted to reduce interference from fluffy hair and movement on the signal. Subsequently, based on the selected target drive current value and target wave band, the light source of the photoelectric sensor is driven to emit light waves.

S104, the optical signals received by the detector of the photoelectric sensor are collected, and the heart rate value of the object is detected based on the frequency value of the optical signal.

In one example, the above detection of the heart rate value of the object based on the frequency value of the optical signal includes: performing band-pass filtering on the optical signal to obtain the filtered signal; selecting the K frequency point signals with the highest amplitude values from the filtered signal; performing frequency multiplication interference removal processing on the K frequency point signals to obtain M frequency point signals; selecting effective frequency points from the M frequency point signals; converting the frequency values of the selected effective frequency points into the heart rate value of the object; where K and M are both positive integers.

The step of electing effective frequency points from the above M frequency point signals includes: identifying whether there is an interval with continuous frequency values among the M frequency point signals; if one or more intervals with continuous frequency values exist, then taking multiple frequency points in the interval with the highest frequency value as multiple effective frequency points; the above step of converting the frequency values of the selected effective frequency points into the heart rate value of the object includes: performing weighted average processing on the frequency values of the multiple effective frequency points to obtain the average frequency; and converting the average frequency into the heart rate value of the object.

Furthermore, if there are no intervals with continuous frequency values, then all M frequency point signals are taken as effective frequency points; weighted average processing is performed on the frequency values of the M frequency point signals to obtain the average frequency; the average frequency is converted into the heart rate value.

Specifically, the photoelectric sensor is a photoplethysmography sensor. The optical signal obtained is the PPG (Photoplethysmography) signal. After band-pass filtering and Fast Fourier Transform (FFT), due to the influence of dense hair, the frequency-domain data of the FFT exhibits two types of interference signal characteristics, including harmonic interference and adjacent-frequency drift. Therefore, a supervised machine learning approach is adopted here, constructing a selection algorithm based on a decision tree model to isolate the effective frequency signals. The logic and algorithms for frequency signal separation and determination mainly include the following:

from the FFT frequency-domain data of the PPG signals, the top k (where k is typically set to 5-10) frequencies with the highest amplitude values are selected, forming a set FrqTopK. That is, FrqTopK: {f₁@a₁, f₂@a₂, . . . , fₖ@aₖ}.

The minimum frequency is extracted from the set FrqTopK, denoted as FrqTKMin. For each fₙ belonging to FrqTopK, if fₙ>FrqTKMin*2, then fadjₙ=fₙ/2 is taken, where fadjₙ is rounded to the nearest integer. All adjusted fadjₙ are updated into the set FrqTopK, with the updated set denoted as FrqTopK_{adj}, thereby eliminating harmonic interference.

The central frequency of FrqTopK_{adj} is calculated using a weighting method, which may include amplitude, energy, rank weighting, or arithmetic mean. After rounding down, the central frequency is denoted as f_{kMid}.

Whether the f₁˜fₖ of Fr_{q Top K}_{_{adj}} falls within the frequency interval of [f_{kMid}−OFD, f_{kMid}+OFD] is checked one by one, where OFD is typically set to 3-5. Those fₙ that appear within the interval are retained and those that exceed it are discarded All retained f₁˜fₖ form a set, denoted as frqTKA_{filtered}: {f₁@a₁, f₂@a₂, . . . , fₘ@aₘ}, where m=<k.

Whether the f₁˜fₘ set in the set frqTKA_{filtered} contains numerically consecutive intervals is checked. If at least one consecutive interval is included, the average frequency of the weighted frequency of the consecutive interval with the largest f value is taken; if there are no consecutive intervals, the average frequency of the weighted frequency of all f₁˜fₘ is taken.

Weighting methods include amplitude, energy, or simple arithmetic averaging, etc., to correct for adjacent frequency drift. Taking amplitude-weighted averaging as an example, the average frequency algorithm is:

$${f}_{wavg} = {\sum }_{i = 1}^{m} {a}_{i} {f}_{i} / {\sum }_{i = 1}^{m} {a}_{i} .$$

Here, f_{wavg} is the average frequency, aᵢ represents the amplitude of the i^{th} signal, and fᵢ represents the frequency of the i^{th} signal.

Validation test: when the device is worn on the neck or shoulder of an animal with dense hair, while simultaneously using a stethoscope to monitor the animal's actual heart rate for comparison, the above algorithm can detect the heart rate waveform of animals with dense hair and varying hair colors, and accurately analyze and obtain precise heart rate values from the waveform data of the PPG signal.

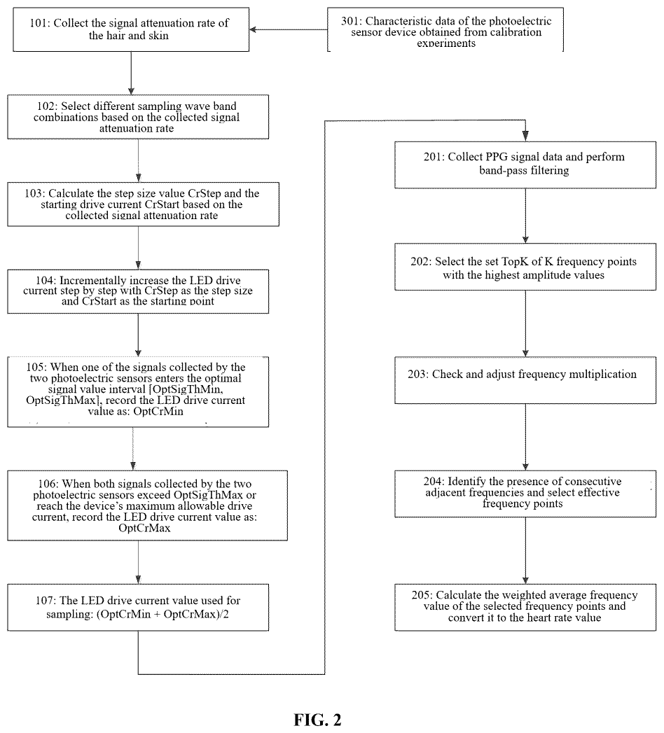

For the adaptive photoelectric heart rate detection method based on hair characteristics described in the above embodiment, a specific example is given below, as shown in FIG. 2:

Here, steps 101-107 are the adaptive light adjustment subprocess; steps 201-205 are the sampling decision tree subprocess capable of eliminating harmonic and adjacent frequency interference; and step 301 is the experimentally measured component characteristic data.

In actual animal experiments, cats, dogs, and rabbits with different hair colors (light, light brown, dark brown, black) and varying hair lengths (5-50 mm) were selected as test objects, with the device worn on the neck or shoulder. The results showed:

Under light to dark brown hair conditions, using the adaptive photoelectric heart rate detection method based on hair characteristics described in this embodiment, the device achieved a heart rate detection rate of 95% and an accuracy of approximately 90%;

Under black hair and lengths less than 40 mm, the heart rate accuracy remained above 80%;

Under all experimental conditions, respiratory rate signals could be simultaneously captured, demonstrating excellent applicability.

Compared to traditional human heart rate wristbands/watches, under the same experimental conditions, only about 20% detection rate could be achieved, with accuracy below 15%.

Therefore, the above adaptive photoelectric heart rate detection method based on hair characteristics can achieve:

1. High detection rate: in pet experiments, using the adaptive photoelectric heart rate detection method based on hair characteristics, the device's heart rate detection rate increased from approximately 20% with traditional devices to 95%.

2. High Accuracy: when using stethoscope readings as the gold standard, the adoption of an adaptive photoelectric heart rate detection method based on hair characteristics achieves a measurement accuracy of over 85%; in contrast, using ordinary heart rate wristbands/watches on pets with hair at the neck yields an accuracy of only about 15%.

3. Broad Applicability: under conditions of light to dark brown hair, the accuracy approaches 90%; even with black hair shorter than 40 mm, the accuracy remains above 80%.

4. Expandable Monitoring Metrics: beyond heart rate, it can also accurately monitor respiratory frequency under certain conditions.

In summary, this application provides an adaptive photoelectric heart rate detection method based on hair characteristics. By leveraging multi-wave band light source differential modeling, photodiode array zonal correction, hair characteristic modeling, and AI noise suppression technology, it significantly improves the detection rate and accuracy of heart rate signals in scenarios with pet hair coverage, demonstrating strong practical application value and market potential.

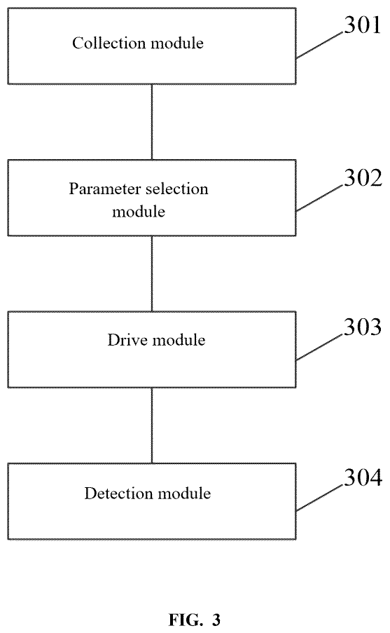

This application also provides an adaptive photoelectric heart rate detection device based on hair characteristics. As shown in FIG. 3, the device includes a collection module 301, a parameter selection module 302, a drive module 303 and a detection module 304. The collection module 301 is used to gather the signal attenuation rate of the hair of the object wearing the photoelectric sensor; the parameter selection module 302 selects the target wave band of the photoelectric sensor's light source based on the signal attenuation rate and determines the target drive current value for the photoelectric sensor; the drive module 303 drives the light source of the photoelectric sensor to emit light waves according to the target drive current value and target wave band; the detection module 304 collects the optical signals received by the detector of the photoelectric sensor and detects the heart rate value of the object based on the frequency value of the optical signals.

For specific details on the adaptive photoelectric heart rate detection device based on hair characteristics, refer to the aforementioned description of the adaptive photoelectric heart rate detection method based on hair characteristics, which will not be repeated here. Each module in the aforementioned device can be implemented wholly or partially through software, hardware, or a combination thereof. These modules may be embedded in or independent of the processor of a wearable electronic device in hardware form, or stored in the memory of the wearable electronic device in software form for the processor to execute the corresponding operations of each module.

The embodiment of this application provides a storage medium, specifically a computer-readable storage medium, on which a computer program is stored. When the computer program is executed by a processor, it implements the adaptive photoelectric heart rate detection method based on hair characteristics as described in any one of the aforementioned embodiments. The computer-readable storage medium includes, but is not limited to, any type of disk (including floppy disks, hard disks, optical disks, CD-ROMs, and magneto-optical disks), ROM (Read-Only Memory), RAM (Random Access Memory), EPROM (Erasable Programmable Read-Only Memory), EEPROM (Electrically Erasable Programmable Read-Only Memory), flash memory, magnetic cards, or optical cards. In other words, the storage device includes any medium that can store or transmit information in a readable form by a device (e.g., a computer or mobile phone), such as read-only memory, magnetic disks, or optical disks.

The embodiment of this application also provides a wearable electronic device. The wearable electronic device is equipped with a reflective photoelectric sensor, which includes a microprocessor, memory, and a computer program stored in the memory and executable on the processor. When the processor executes the computer program, it implements the adaptive photoelectric heart rate detection method based on hair characteristics as described in any one of the aforementioned embodiments.

The above-described embodiments merely represent several implementations of this application, and their descriptions are relatively specific and detailed. However, they should not be construed as limiting the scope of the patent. It should be noted that for those of ordinary skill in the art, various modifications and improvements can be made without departing from the concept of this application, all of which fall within the protection scope of this application. Therefore, the protection scope of this patent shall be determined by the appended claims.

What is claimed is:

1. An adaptive photoelectric heart rate detection method based on hair characteristics, comprising the following steps of:
collecting a signal attenuation rate of the hair of an object wearing a photoelectric sensor;
selecting a target wave band of a light source of the photoelectric sensor based on the signal attenuation rate, and selecting a target drive current value of the photoelectric sensor based on the signal attenuation rate;
driving the light source of the photoelectric sensor to emit light waves according to the target drive current value and the target wave band; and
collecting optical signals received by a detector of the photoelectric sensor, and detecting a heart rate value of the object based on a frequency value of the optical signals.

2. The adaptive photoelectric heart rate detection method according to claim 1, wherein the step of collecting a signal attenuation rate of the hair of an object wearing a photoelectric sensor comprises:
providing the photoelectric sensor with a certain light wavelength and a drive current, and collecting a signal intensity received by the photoelectric sensor;
obtaining a standard reflected signal intensity; and
determining the signal attenuation rate based on the collected signal intensity and the standard reflected signal intensity.

3. The adaptive photoelectric heart rate detection method according to claim 1, wherein the signal attenuation rate is calculated by providing the photoelectric sensor with a green light wave band and a certain drive current;
the step of selecting a target wave band of a light source of the photoelectric sensor based on the signal attenuation rate comprises:
when the signal attenuation rate is less than a first set value, selecting the target wave band of the light source of the photoelectric sensor as a wave band combining green light with an infrared wavelength, prioritizing green light;
when the signal attenuation rate is greater than or equal to the first set value and less than a second set value, selecting the target wave band of the light source of the photoelectric sensor as a wave band combining infrared with a green light wavelength, prioritizing infrared;
when the signal attenuation rate is greater than or equal to the second set value and less than a third set value, selecting the target wave band of the light source of the photoelectric sensor as a wave band combining infrared with a red light wavelength, prioritizing infrared; and
when the signal attenuation rate is greater than or equal to the third set value, selecting the target wave band of the light source of the photoelectric sensor as a wave band combining infrared with a red light wavelength, prioritizing infrared, wherein when the signal attenuation rate is greater than or equal to the third set value, the optical signals of the photoelectric sensor require repeated sampling for multiple times; and
wherein the first set value is greater than 0 and less than the second set value, the second set value is less than the third set value, and the third set value is less than 1.

4. The adaptive photoelectric heart rate detection method according to claim 1, wherein there are multiple photoelectric sensors, and the step of selecting a target drive current value of the photoelectric sensor based on the signal attenuation rate comprises:
obtaining a lower limit value and an upper limit value of the signal intensity of the photoelectric sensor;
obtaining a starting drive current value and a step size value of the drive current of the photoelectric sensor;
providing the drive current to each photoelectric sensor based on the starting drive current value, and collecting the signal intensity of each photoelectric sensor, wherein if the signal intensity of any photoelectric sensor is not greater than the lower limit value, the drive current of each photoelectric sensor is gradually increased according to the step size value until the signal intensity of at least one photoelectric sensor exceeds the lower limit value, and a drive current value at this time is recorded as OptCrMin;
continuing to gradually increase the drive current of each photoelectric sensor according to the step size value until the signal intensity of all photoelectric sensors is greater than the lower limit value and less than the upper limit value, and recording the drive current value at this time as OptCrMax; and
selecting the target drive current value based on the OptCrMin and the OptCrMax.

5. The adaptive photoelectric heart rate detection method according to claim 4, wherein there are two photoelectric sensors, and the step of selecting the target drive current value based on the OptCrMin and the OptCrMax comprises:
selecting a mean value of the OptCrMin and the OptCrMax as the target drive current value.

6. The adaptive photoelectric heart rate detection method according to claim 1, wherein the step of detecting a heart rate value of the object based on a frequency value of the optical signals comprises:
performing band-pass filtering on the optical signals to obtain filtered signals;
selecting K frequency point signals with a highest amplitude value from the filtered signal;
performing frequency multiplication interference removal processing on the K frequency point signals to obtain M frequency point signals;
screening out effective frequency points from the M frequency point signals; and
converting frequency values of the screened effective frequency points into the heart rate value of the object;
where K and M are both positive integers.

7. The adaptive photoelectric heart rate detection method according to claim 6, wherein the step of screening out effective frequency points from the M frequency point signals comprises:
identifying whether there is an interval with consecutive frequency values among the M frequency point signals;
if one or more intervals with consecutive frequency values exist, selecting multiple frequency points in the interval with a highest frequency value as multiple effective frequency points;
the step of converting frequency values of the screened effective frequency points into the heart rate value of the object comprises:
performing weighted average processing on the frequency values of multiple effective frequency points to obtain an average frequency; and
converting the average frequency into the heart rate value of the object.

8. The adaptive photoelectric heart rate detection method according to claim 7, wherein the method further comprises:
if no intervals with consecutive frequency values exist, treating all M frequency point signals as the effective frequency points;
performing weighted average processing on the frequency values of the M frequency point signals to obtain the average frequency; and
converting the average frequency into the heart rate value of the object.

9. A wearable electronic device, wherein the wearable electronic device is equipped with a reflective photoelectric sensor comprising a microprocessor and a memory, as well as a computer program stored in the memory and executable on a processor, wherein the processor, when executing the computer program, implements the steps of the method according to claim 1.

10. A storage medium, having a computer program stored thereon, wherein the computer program, when executed by a processor, implements the steps of the method according to claim 1.
